(19) United States
(12) Patent Application Publication
(10) Pub. No.: US 2024/0156627 A1
(43) Pub. Date: May 16, 2024

(54) APPARATUSES AND METHODS FOR EXPANDABLE BRANCHING STENT PROSTHESES

(71) Applicant: Merit Medical Systems, Inc., South Jordan, UT (US)

(72) Inventors: Michael Adams, Bluffdale, UT (US); Christopher Cindrich, Highland, UT (US); John Hall, Bountiful, UT (US); Joanne Hislop, American Fork, UT (US); Lindsay Koren, South Jordan, UT (US); Yuma Medina, South Jordan, UT (US); Wayne Mower, Bountiful, UT (US); Jason Wiersdorf, West Jordan, UT (US)

(21) Appl. No.: 18/509,005

(22) Filed: Nov. 14, 2023

Related U.S. Application Data
(60) Provisional application No. 63/383,878, filed on Nov. 15, 2022.

(51) Int. Cl. A61F 2/954; A61F 2/958; A61F 2/966

(52) U.S. Cl. CPC A61F 2/954; A61F 2/958; A61F 2/966; A61B 2017/00358

(57) ABSTRACT

Expandable branching stent prostheses and methods of deployment for the same are discussed herein. An expandable branching stent prosthesis may include a trunk portion, a first branching portion, and a second branching portion that are for deployment at, respectively, a trunk, a first branch, and a second branch of a branching implant site of an anatomical system. Methods for deploying the expandable branching stent prostheses include delivering a deployment system including the expandable branding stent prostheses, a balloon, a distal end of a floss wire, and in some cases a sock to the branching implant site; connecting, to the floss wire, a distal end of a snare wire delivered to the branching implant site via the second branch, and using the two floss wires to manipulate the deployment system to effectuate the deployment. In some cases, the sock may be pulled down over the balloon after the deployment.

### CROSS-REFERENCE TO RELATED APPLICATIONS

This application claims priority to U.S. Provisional Patent Application No. 63/383,878 filed on Nov. 15, 2022 and titled “Apparatuses and Methods for Expandable Branching Stent Prostheses,” which is hereby incorporated by reference in its entirety.

### TECHNICAL FIELD

The present application relates to medical devices including stent prostheses, deployment devices, and methods of using the same.

### BRIEF DESCRIPTION OF THE SEVERAL VIEWS OF THE DRAWINGS

The embodiments disclosed herein will become more fully apparent from the following description and appended claims, taken in conjunction with the accompanying drawings. The drawings depict only typical embodiments, which embodiments will be described with additional specificity and detail in connection with the drawings in which:

FIG. 1 illustrates a perspective view of an expandable branching stent prosthesis, according to embodiments herein.

FIG. 2 illustrates a deployed expandable branching stent prosthesis, according to embodiments herein.

FIG. 3 illustrates a deployment system that uses an expandable branching stent prosthesis, according to embodiments herein.

FIG. 4 illustrates a deployment system configured for use with an expandable branching stent prosthesis, according to embodiments herein.

FIG. 5 illustrates a first deployment stage for a deployment system used to deploy an expandable branching stent prosthesis at a branching implant site, according to embodiments herein.

FIG. 6A illustrates a subsequent deployment stage for the deployment system, according to embodiments herein.

FIG. 6B through FIG. 6E illustrate further subsequent deployment stages for a first deployment system, according to embodiments herein.

FIG. 7A and FIG. 7B illustrate further subsequent deployment stages for a second deployment system, according to embodiments herein.

FIG. 8 illustrates a method according to embodiments disclosed herein.

### DETAILED DESCRIPTION

Delivery catheter systems may be configured to deliver one or more medical appliances or systems to a location within a patient's body and deploy the medical appliance or system within the patient's body. For example, such a delivery catheter system may be configured to be advanced from an insertion site at the outside of an anatomical system to a treatment location within the anatomical system. For example, a delivery catheter system may be configured to be advanced through bends, turns, or other structures within the anatomy of the vasculature.

A stent prosthesis may be disposed within a portion of the delivery catheter system (e.g., as or as part of a medical appliance or system) such that a practitioner may deploy the stent prosthesis from a distal end of the delivery catheter system through manipulation of one or more components of a handle assembly of the delivery catheter system.

Stent prostheses may be deployed in various body lumens for a variety of purposes. Stent prostheses may be deployed, for example, in the arterial system for a variety of therapeutic purposes including the treatment of occlusions within the lumens of that anatomical system. It will be appreciated that the current disclosure may be applicable to stent prostheses designed for the central venous system, peripheral vascular system, abdominal aortic aneurism treatment, bronchial system, esophageal system, biliary system, or any other system of the human body. Further, the present disclosure may equally be applicable to other prosthesis such as grafts.

Accordingly, it will be understood that while specific examples recited herein may refer to deployment of cardiovascular stent prostheses within a cardiovascular system, analogous concepts and devices may be used in/with various other anatomical systems of the body, including for placement and deployment of medical appliances in the gastrointestinal tract (including, for example, within the esophagus, intestines, stomach, small bowel, colon, and biliary duct); the respiratory system (including, for example, within the trachea, bronchial tubes, lungs, nasal passages, and sinuses); or any other location within the body, both within bodily lumens (for example, the ureter, the urethra, etc.) and within other bodily structures.

The phrases “coupled to” and “in communication with” refer to any form of interaction between two or more entities, including mechanical, electrical, magnetic, electromagnetic, fluid, and thermal interaction. Two components may be coupled to or in communication with each other even though they are not in direct contact with each other. For example, two components may be coupled to or in communication with each other through an intermediate component.

The directional terms “proximal” and “distal” are used herein to refer to opposite locations relative to a medical device in use by a practitioner. The proximal end of the device is defined as the end of the device closest to the practitioner when the device is in use by the practitioner. The distal end is the end opposite the proximal end.

Embodiments may be understood by reference to the drawings, wherein like parts are designated by like numerals throughout. It will be understood by one of ordinary skill in the art having the benefit of this disclosure that the components of the embodiments, as generally described and illustrated in the figures herein, could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

It will be appreciated that various features are sometimes grouped together in a single embodiment, figure, or description thereof for the purpose of streamlining the disclosure. Many of these features may be used alone and/or in combination with one another.

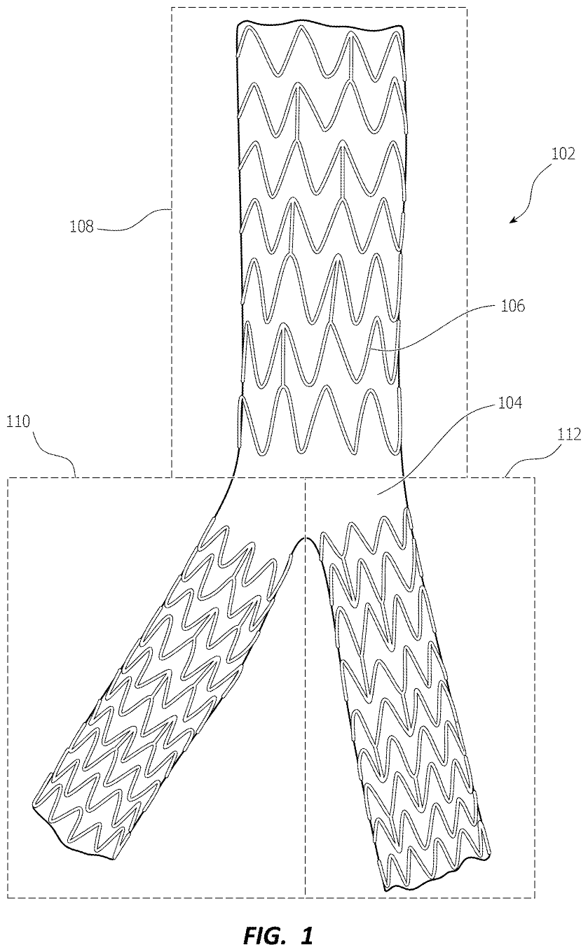

FIG. 1 illustrates a perspective view of an expandable branching stent prosthesis 102, according to embodiments herein. The expandable branching stent prosthesis 102 of FIG. 1 is illustrated in a deployed (expanded) position.

In the illustrated embodiment, the expandable branching stent prosthesis 102 is partially composed of a wire or laser cut stent 106. The wire or laser cut stent 106 is configured to be expandable to the illustrated deployed position from an unexpanded/un-deployed configuration (e.g., via the inflation of one or more balloons from within the expandable branching stent prosthesis 102) after the expandable branching stent prosthesis 102 is delivered to a desired location. The wire or laser cut stent 106 may be formed of any suitable material, such as nickel-titanium alloy, stainless steel, cobalt-chromium, platinum, polymers, etc. The wire or laser cut stent 106 may have a zig-zag pattern, a wave pattern, or any other suitable pattern. The wire or laser cut stent 106 may be pre-formed or formed corresponding to a tubular body 104. The material, pattern, and wire diameter of a wire or laser cut stent 106 that is a wire stent, or the wall thickness and strut width of a wire or laser cut stent 106 that is a laser cut stent, may be configured to provide a chronic radial outwardly directed force and a resistance to a radial inwardly directed force. For a non-self-expanding design the deployed wire or laser cut stent 106 may be configured to provide a radial stiffness and radial strength to resist a local or radial inwardly directed force.

The expandable branching stent prosthesis 102 further includes the tubular body 104. The tubular body 104 may be formed of a variety of materials and/or layers of materials, including biocompatible materials that are resistant to passage of fluid through a wall of the tubular body 104. For example, the tubular body 104 may be formed of polyethylene terephthalate, polyurethane, silicone rubber, nylon, fluoropolymer, polyester, etc. A thickness of the wall may range from about 0.025 mm to about 0.5 mm.

In certain embodiments, the wall of the tubular body 104 may be impermeable to tissue cell ingrowth into and/or tissue cell migration through the wall, for example, to prevent or discourage stenosis of the tubular body 104. Additionally or alternatively, in some embodiments, the wall of the tubular body 104 can be impermeable to fluid such that fluid is prevented from leaking from the inside of the expandable branching stent prosthesis 102 to the exterior of the expandable branching stent prosthesis 102 and into surrounding tissue. In some embodiments an interior surface of the wall may include serially deposited fibers of polytetrafluoroethylene (PTFE) to resist fibrin deposition and platelet adhesion on the surfaces.

Note that in embodiments herein, stent prostheses are illustrated as having tubular bodies (such as the tubular body 104 of FIG. 1). However, it will be understood that expandable branching stent prostheses using a wire scaffold, framework, or stent without a cover or other tubular body fall within the scope of the disclosure.

As illustrated herein, the expandable branching stent prosthesis 102 may be for deployment at a branching implant site within a body having a trunk, a first branch, and a second branch. Accordingly, the expandable branching stent prosthesis 102 includes a trunk portion 108, a first branching portion 110, and a second branching portion 112. For deployment to the expandable branching stent prosthesis 102 at the branching implant site, the trunk portion 108 of the expandable branching stent prosthesis 102 is used in/at the trunk of the branching implant site, the first branching portion 110 is deployed in/at the first branch of the branching implant site, and the second branching portion 112 is deployed in/at the second branch of the branching implant site.

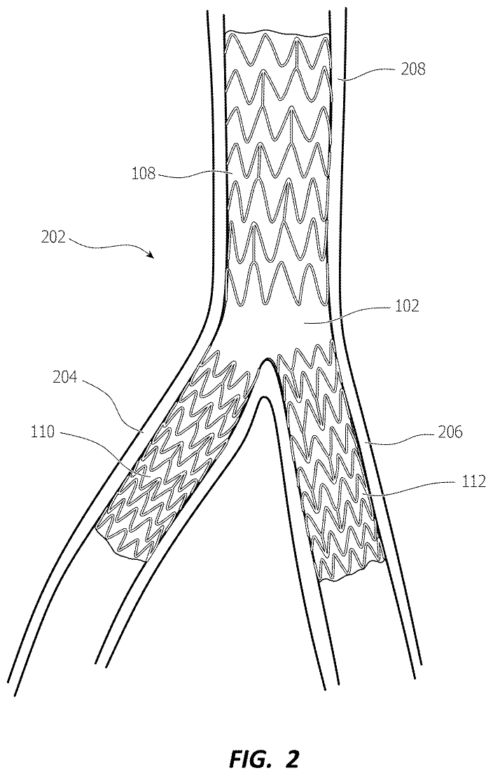

FIG. 2 illustrates a deployed expandable branching stent prosthesis 102, according to embodiments herein. In this expanded/deployed configuration, the expandable branching stent prosthesis 102 provides stenting at/through a branching implant site 202 within the anatomical system in which the expandable branching stent prosthesis 102 is deployed.

The branching implant site 202 may be a portion of an anatomical system that includes the first branch 204, the second branch 206, and the trunk 208 (and that, e.g., communicates fluids through these). In some cases, the branching implant site 202 may be the aorto-illiac bifurcation in the arterial system. In the illustrated deployed configuration, the trunk portion 108 of the expandable branching stent prosthesis 102 is deployed at/within the trunk 208 of the branching implant site 202, the first branching portion 110 of the expandable branching stent prosthesis 102 is deployed at/within the first branch 204 of the branching implant site 202, and the second branching portion 112 of the expandable branching stent prosthesis 102 is deployed at/within the second branch 206 of the branching implant site 202.

In some embodiments, the expandable branching stent prosthesis 102, once deployed, provides an appropriate channel for desired liquid flow through the branching implant site 202. It may be that the region around the branching implant site 202 is diseased, misshapen, and/or damaged, and that the deployment of the expandable branching stent prosthesis 102 in the illustrated manner can correct and/or ameliorate attendant issues.

As illustrated, the branching implant site 202 of FIG. 2 is used within an anatomical system that is a cardiovascular system. However, it should be noted that while examples herein describe (and figures herein illustrate) the deployment of expandable branching stent prostheses within cardiovascular systems, the cardiovascular anatomical context is given by way of example and not by way of limitation. It will be understood that deployment systems analogous to those described in relation to disclosure herein may be used to deploy appropriate expandable branching stent prostheses with/at branching implant sites of other anatomical systems, and that corresponding methods for using such deployment systems to deploy corresponding expandable branching stents that are analogous to those methods described herein could be used in those other anatomical contexts.

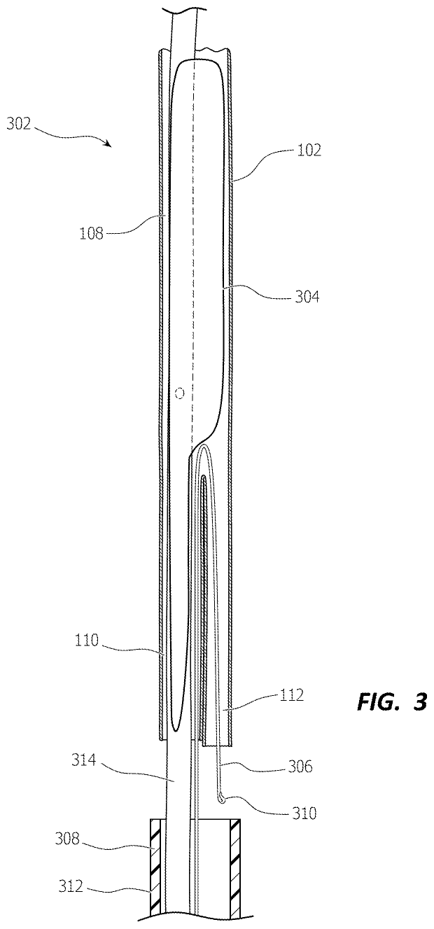

FIG. 3 illustrates a deployment system 302 configured for use with an expandable branching stent prosthesis 102, according to embodiments herein. As illustrated, the deployment system 302 may include an expandable branching stent prosthesis 102, a balloon 304, and a distal end of a floss wire 306.

The expandable branching stent prosthesis 102 is illustrated in FIG. 3 in a collapsed (non-deployed configuration), but is otherwise as described in relation to FIG. 1.

A delivery catheter system 308 may be used to deliver the deployment system 302 to a desired location. The deployment system 302 may be mounted on a catheter lumen 314 of the delivery catheter system 308 by passing the catheter lumen 314 through the first branching portion 110 and the trunk portion 108 of the expandable branching stent prosthesis 102. A catheter sleeve 312 of the deployment system 302 may initially cover/surround the deployment system 302 in order to hold the deployment system 302 in place on the catheter lumen 314 and to prevent snagging of the deployment system 302 during placement. Once the deployment system 302 has been located in the desired location, the catheter sleeve 312 may be retracted back along the catheter lumen 314 to expose the deployment system 302 (as illustrated in FIG. 3).

In alternative cases, it may be that the deployment system 302 is not covered by the catheter sleeve 312 during placement. In such a case, the catheter lumen 314 on which the deployment system 302 is mounted (e.g., via crimping) would be manipulated to extend outward from the delivery catheter system 308 until the catheter lumen 314 is located at the desired location (as illustrated in FIG. 3).

The balloon 304 is arranged within the expandable branching stent prosthesis 102. The balloon 304 may be attached to the catheter lumen 314 along its length, and may be inflated and/or deflated at the option of a practitioner operating the delivery catheter system 308 via a connection between the balloon 304 and the catheter lumen 314 that communicates fluid between the catheter lumen 314 and the balloon 304. As can be seen, due to the shape of the balloon 304 and its placement/arrangement within the expandable branching stent prosthesis 102, when the balloon 304 is inflated the balloon 304 expands the trunk portion 108 and the first branching portion 110 of the expandable branching stent prosthesis 102 for deployment.

The distal end of the floss wire 306 passes through the first branching portion 110 of the expandable branching stent prosthesis 102 and out the second branching portion 112 of the expandable branching stent prosthesis 102, as illustrated. The distal end of the floss wire 306 passes along the outside of the balloon 304 in such a manner that there is substantially no translational dependency between the floss wire 306 and the balloon 304 (the floss wire may be advanced or retracted through the expandable branching stent prosthesis 102 without necessarily repositioning the balloon 304). The distal end of the floss wire 306 terminates in a catch feature 310 that is configured to interact with a snare feature of a snare wire, in the manner described herein.

When deployed within an anatomical system, the floss wire 306 may extend back through a branch of a branching implant site for planned deployment of the expandable branching stent prosthesis 102 of the deployment system 302 and run to/through an insertion site through which a practitioner has delivered the deployment system 302 to the body (e.g., using a delivery system (such as, e.g., a delivery catheter system) that includes the deployment system 302).

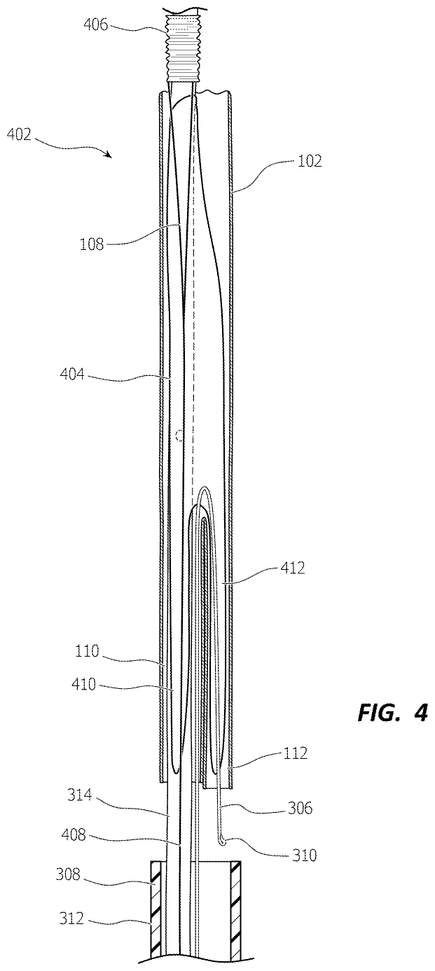

FIG. 4 illustrates a deployment system 402 that uses an expandable branching stent prosthesis 102, according to embodiments herein. As illustrated, the deployment system 402 may include an expandable branching stent prosthesis 102, a balloon 404, a distal end of a floss wire 306, a sock 406, and one or more sock wire(s) 408.

The expandable branching stent prosthesis 102 is illustrated in FIG. 4 in a collapsed (non-deployed configuration), but is otherwise as described in relation to FIG. 1.

Similarly to the deployment system 302 of FIG. 3, the delivery catheter system 308 may be used to deliver the deployment system 402. As in FIG. 3, the catheter lumen 314 may be passed through the first branching portion 110 and the trunk portion 108 of the expandable branching stent prosthesis 102, and the catheter sleeve 312 of the deployment system 302 may initially cover the deployment system 402 and hold the deployment system 402 in place on the catheter lumen 314. Once the deployment system 302 has been located in the desired location, the catheter sleeve 312 may be retracted back along the catheter lumen 314 to expose the deployment system 402 (as illustrated in FIG. 4).

With further similarity to FIG. 3, in alternative cases, it may be that the deployment system 402 is not covered by the catheter sleeve 312 during placement. In such a case, the catheter lumen 314 on which the deployment system 402 is mounted (e.g., via crimping) would be manipulated to extend outward from the delivery catheter system 308 until the catheter lumen 314 is located at the desired location (as illustrated in FIG. 3).

The balloon 404 is arranged within the expandable branching stent prosthesis 102. The balloon 404 may be attached to the catheter lumen 314, and may be inflatable and/or deflatable at the option of a practitioner operating the delivery catheter system 308 via a connection between the balloon 404 and the catheter lumen 314 that can communicate fluid between the catheter lumen 314 and the balloon 404. As can be seen (and alternatively to the balloon 304 of FIG. 3), due to the forked shape of the balloon 404 and its placement/arrangement within the expandable branching stent prosthesis 102, when the balloon 404 is inflated balloon 404 expands the trunk portion 108, the first branching portion 110, and the second branching portion 112 of the expandable branching stent prosthesis 102 for deployment.

It is noted that the attachment of the balloon 404 to the catheter lumen 314 may occur along the first balloon leg 410 of the balloon 404. It may be that the second balloon leg 412 is not so attached (such that the second balloon leg 412 can be located/arranged within the second branching portion 112 of the expandable branching stent prosthesis 102 in the manner illustrated).

The floss wire 306 may be as is described in relation to FIG. 3 (e.g., the illustrated distal end of the floss wire 306 passes through the first branching portion 110 of the expandable branching stent prosthesis 102 and out the second branching portion 112 of the expandable branching stent prosthesis 102 and terminates in the catch feature 310, and where the floss wire 306 has translational independence from the balloon 304). Further, as described in relation to FIG. 3, the floss wire 306 may extend back through a branch of a branching implant site for planned deployment of the expandable branching stent prosthesis 102 of the deployment system 302 and run to/through an insertion site.

The sock 406 may be a rolled up sleeve that surrounds the catheter lumen 314. As illustrated, when in the rolled up configuration, the sock 406 is in a distal position relative to the balloon 404. The sock 406 may be configured to be pulled down to an unrolled configuration that holds the balloon 404 against the catheter lumen 314 (e.g., to facilitate retraction of the catheter lumen 314 and/or delivery catheter system 308 after the balloon 404 has been used to extend/deploy the expandable branching stent prosthesis 102, in the manner described herein). Accordingly, one or more sock wire(s) 408 attached to the bottom of the sock 406 may be used to cause the sock to pull over the balloon 404 (e.g., cause the sock 406 to translate from the rolled up configuration to the unrolled configuration) in the event that the sock wire(s) 408 is/are held stationary while the catheter lumen 314 (to which the top of the sock 406) is attached) and/or delivery catheter system 308 is advanced (translated in the upward direction shown in FIG. 4).

In some embodiments, the floss wire 306 may extend through the center of the second balloon leg 412. This configuration keeps the floss wire 306 centered in the delivery catheter system 308, which, in turn, allows the introducer sheath to be more easily advanced up the second branch 206 and over the second branching portion 112. For example, in use, doctors may prefer feeding an introducer sheath up the second branch 206 and over the second branching portion 112 to make pathway for the second branching portion 112 rather than extending the second branching portion 112 down into the second branch 206 with no protection, as the second branching portion 112 may catch on calcification within the second branch 206.

In some embodiments, the sock may be made of polytetrafluoroethylene (PTFE).

Methods for using deployment systems described herein to deploy expandable branching stent prostheses to a branching implant site within a body are now described.

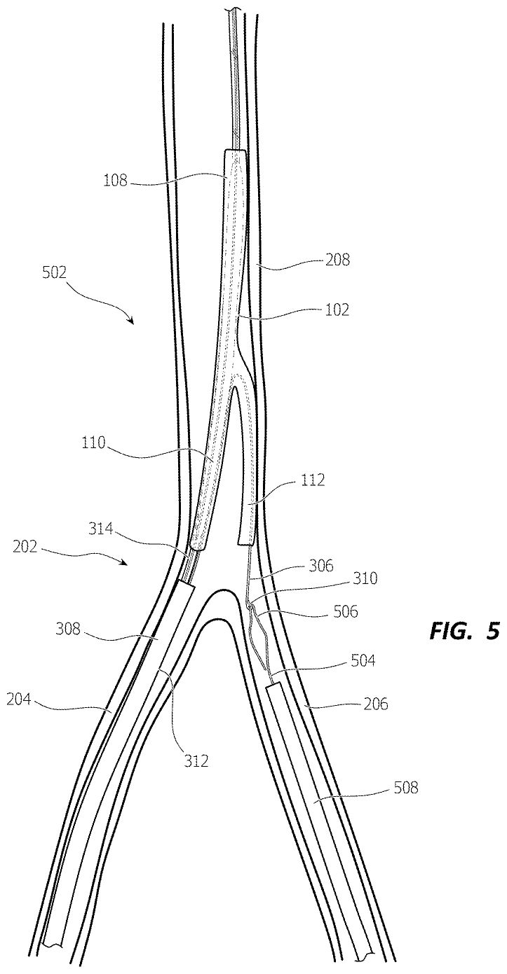

FIG. 5 illustrates a first deployment stage for a deployment system 502 used to deploy an expandable branching stent prosthesis 102 at a branching implant site 202, according to embodiments herein.

The deployment system 502 may be a deployment device as described herein (e.g., the deployment system 302 of FIG. 3 and/or the deployment system 402 of FIG. 4).

One or more incisions (not illustrated) giving access to a first branch 204 of the branching implant site 202 is made in the body. Then, the delivery catheter system 308 is used to deliver, via the first branch 204, the deployment system 502 to the illustrated location past the first branch 204 and into the trunk 208 of the branching implant site 202.

While advancing up through the first branch 204, the deployment system 502 may be covered by the catheter sleeve 312 of the delivery catheter system 308 to facilitate positioning within the trunk 208. Then, once the desired positioning of the deployment system 502 is achieved, the catheter sleeve 312 may be retracted along the catheter lumen 314 to expose the deployment system 502 at a selected position (e.g., such that the positioning of the deployment system 502 is as illustrated in FIG. 5 (but prior to the use of the snare wire 504 to snare the floss wire 306, to be described below).

Alternatively, the catheter lumen 314 may be advanced forward from the delivery catheter system 308 until the deployment system 502 is located in the desired position (e.g., such that the positioning of the deployment system 502 is as illustrated in FIG. 5 (but prior to the use of the snare wire 504 to snare the floss wire 306, to be described below).

As illustrated, the deployment system 502 is delivered with an orientation on the catheter lumen 314 such that the first branching portion 110 and the second branching portion 112 of the expandable branching stent prosthesis 102 are oriented corresponding to the first branch 204 and the second branch 206 of the branching implant site 202.

It is also noted that the floss wire 306 extends back from the deployment system 502 and to the insertion site corresponding to the incision(s) for access to the first branch 204.

Once the deployment system 502 is in the desired position, one or more incisions (not illustrated) giving access to the second branch 206 of the branching implant site 202 is made in the body. Then, a distal end of a snare wire 504 is delivered to the branching implant site 202 via the second branch 206 (e.g., using a second delivery catheter system 508 through the access incision(s) for the second branch 206. The snare wire 504 may extend back through the second branch 206 of the branching implant site 202 for to/through an insertion site corresponding to the incision(s) for access to the second branch 206.

As illustrated, the distal end of the snare wire 504 includes a snare feature 506 that is configured to interact with the catch feature 310 of the floss wire 306 in order to connect and/or snare the distal end of the floss wire 306 together with the distal end of the snare wire 504 at the branching implant site 202. A practitioner manipulating the floss wire 306 and/or the snare wire 504 from their respective corresponding insertion sites may cause this connecting and/or snaring together to occur.

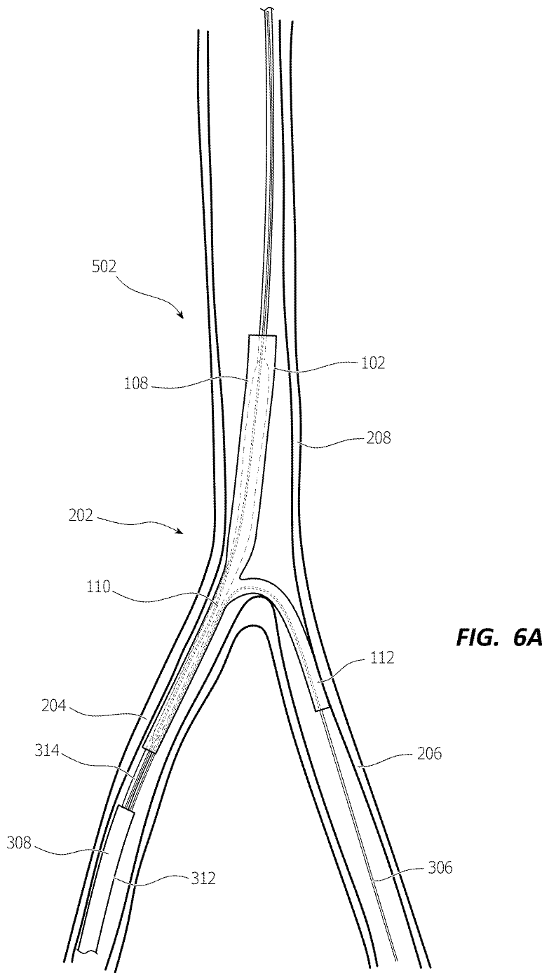

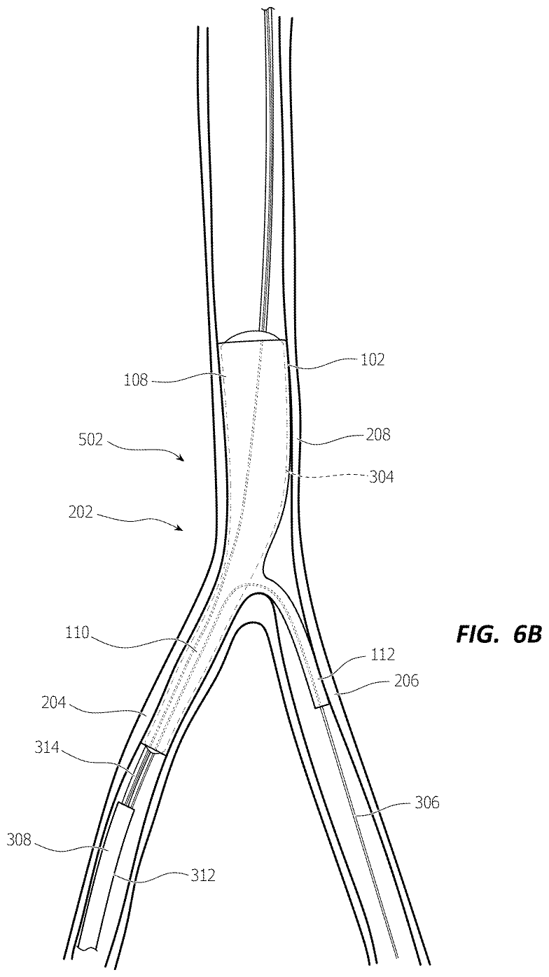

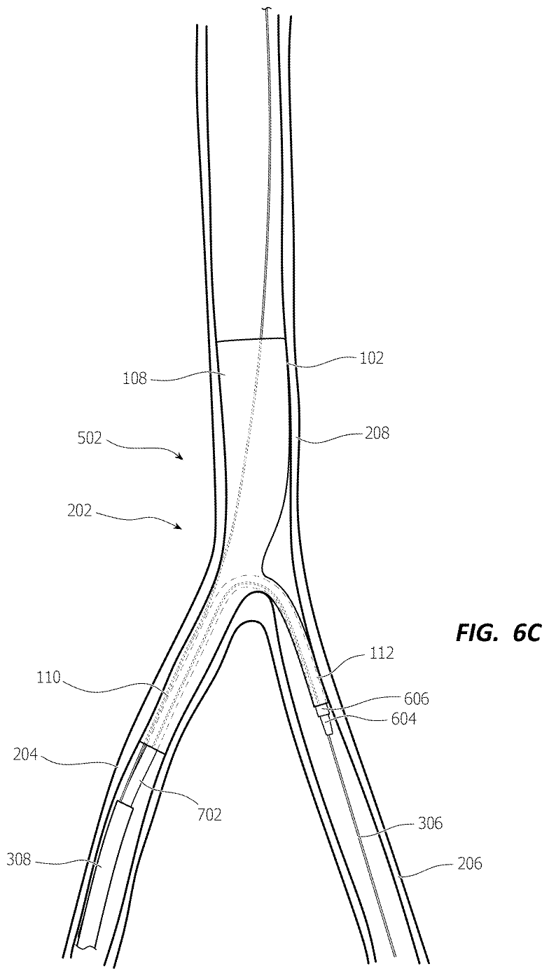

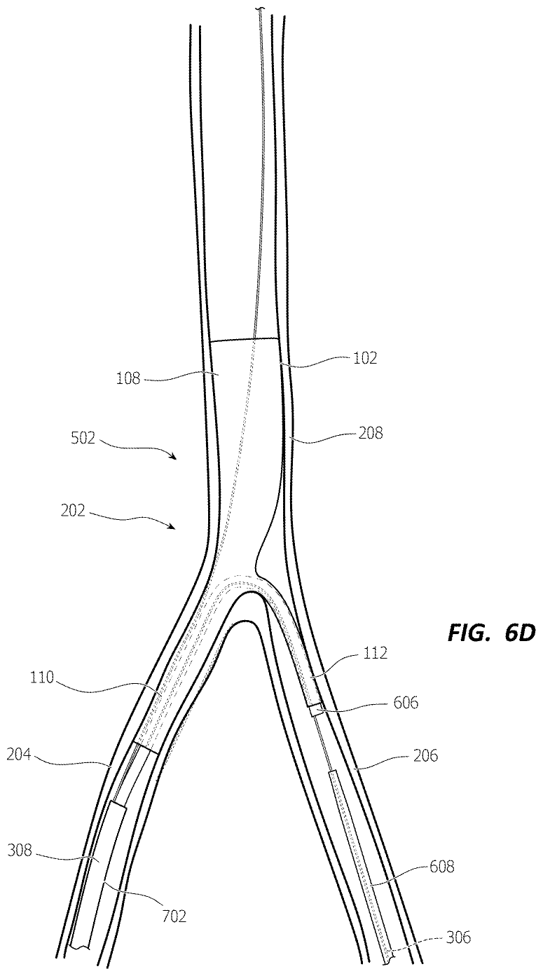

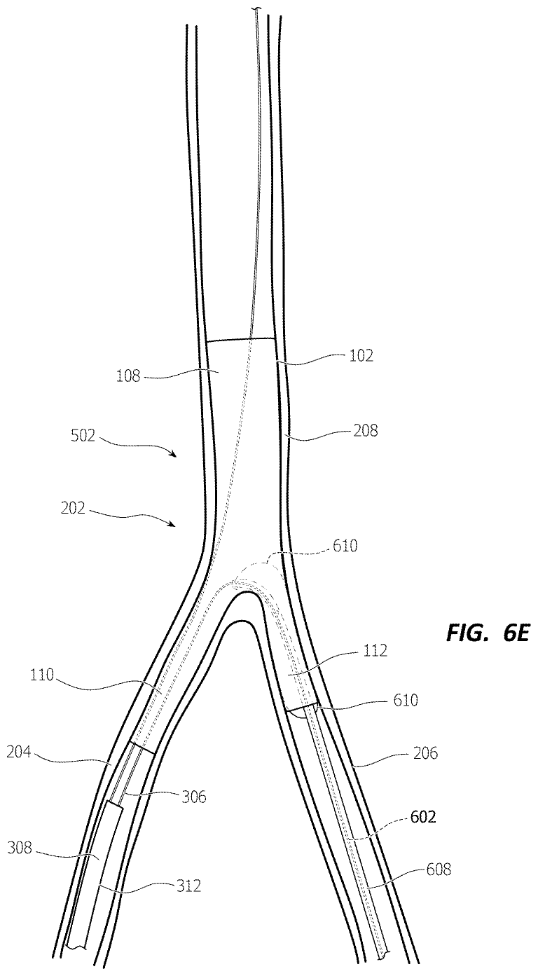

FIG. 6A through FIG. 6E illustrate further subsequent deployment stages for a first deployment system 502, according to embodiments herein. In the embodiment of FIG. 6A through FIG. 6E, the deployment system 502 may be the same as the deployment system 302 discussed in FIG. 3.

According to the embodiments of FIG. 6A through FIG. 6E, after the floss wire 306 and the snare wire 504 are connected together (as illustrated in FIG. 5), the snare wire 504 may be pulled back along the second branch 206 in order to bring the distal end of the floss wire 306 through the second branch 206 and out through the insertion site/incision for the access to the second branch 206, as has been described. FIG. 6A accordingly illustrates that the floss wire 306 has been pulled through in this manner.

FIG. 6A also illustrates an example positioning of the deployment system 502 once a desired seating of the expandable branching stent prosthesis 102 has occurred. The pulling of the floss wire 306 through the second branch 206 may cause the first branching portion 110 of the expandable branching stent prosthesis 102 to become seated along the first branch 204 of the branching implant site 202 and the second branching portion 112 of the expandable branching stent prosthesis 102 to become seated along the second branch 206 of the branching implant site 202. Note that if necessary, after the distal end of the second branch 206 is exposed to the practitioner through the insertion site/incision for the access to the second branch 206, further manipulation of the expandable branching stent prosthesis 102 may be performed using both ends of the floss wire 306 at the respective insertion site for each end in order to achieve the appropriate seating of the first branching portion 110 and the second branching portion 112 of the expandable branching stent prosthesis 102 in this manner. As provided above, a sheath may be advanced up the second branch 206 and/or over the second branching portion 112 to clear a pathway for the second branching portion 112 as the second branching portion 112 is pulled down from the trunk 208 into the second branch 206.

With the deployment system 502 in the illustrated position, it may be that the expandable branching stent prosthesis 102 of the deployment system 502 can be usefully deployed within the branching implant site 202.

With the expandable branching stent prosthesis 102 appropriately seated (e.g., as was discussed in FIG. 6A), the balloon of the deployment system 502 (e.g., as in the balloon 304 illustrated in FIG. 2) may be inflated (e.g., the practitioner may manipulate the delivery catheter system 308 for the balloon 304 to cause the inflation of the balloon 304). The inflation of this balloon expands the trunk portion 108 and the first branching portion 110 of the expandable branching stent prosthesis 102, such that the trunk portion 108 and the first branching portion 110 deploy against the walls of the trunk 208 and the first branch 204 of the branching implant site 202, as illustrated in FIG. 6B. The balloon 304 may then be deflated.

With the expandable branching stent prosthesis 102 partially deployed and the balloon 304 serving no further use, the catheter lumen 314 may be retracted out from the branching implant site 202/out of the body altogether (while the partially deployed expandable branching stent prosthesis 102 is left in place).

Then, as illustrated in FIG. 6C, a dilation device 602 may be delivered up the first branch 204 along the floss wire 306 (e.g., through the delivery catheter system 308).

Due to its travel along the floss wire 306, the dilation device 602 proceeds through the first branching portion 110 of the expandable branching stent prosthesis 102 (which can receive the dilation device 602 because the dilation device 602 is already been expanded/deployed as was described in FIG. 6B) and into (and, e.g., through) the second branching portion 112 of the expandable branching stent prosthesis 102. This causes a dilation (e.g., a partial expansion) of the second branching portion 112 of the expandable branching stent prosthesis 102.

As illustrated, a tip of an inner lumen 604 of the dilation device 602 may be tapered to facilitate the navigation of the dilation device 602 through the expandable branching stent prosthesis 102 in the manner described. The dilation device 602 may be pushed through the expandable branching stent prosthesis 102 until a sheath 606 of the dilation device 602 that surrounds the inner lumen 604 beneath the tip is exposed outside the second branching portion 112 of the expandable branching stent prosthesis 102, as illustrated.

Once the dilation of the second branching portion 112 of the expandable branching stent prosthesis 102 is performed and the sheath 606 is exposed outside the second branching portion 112 of the expandable branching stent prosthesis 102, the inner lumen 604 of the dilation device 602 may then be retracted from the branching implant site 202, leaving the sheath 606 of the dilation device 602 in place.

As illustrated in FIG. 6D, a balloon delivery device 608 may then be delivered through the insertion site corresponding to the second branch 206 and along the snare wire 504 up the second branch 206 of the branching implant site 202 preparatory to insertion into the second branching portion 112 of the expandable branching stent prosthesis 102.

The balloon delivery device 608 includes a balloon 610 (see FIG. 6E) attached to the balloon delivery device 608 and that is inflatable and/or deflatable via balloon delivery device 608 (e.g., via a practitioner's operation of the balloon delivery device 608).

Note that due to the dilation of the second branching portion 112 of the expandable branching stent prosthesis 102 and the presence of the sheath 606, it is feasible to deliver the balloon delivery device 608 into the second branching portion 112 of the expandable branching stent prosthesis 102 by coming up the second branch 206 of the branching implant site 202 along the snare wire 504 and through the sheath 606 (which provides structure/scaffolding for access into the second branching portion 112 of the expandable branching stent prosthesis 102 by the balloon delivery device 608).

The balloon delivery device 608 may be positioned (e.g., by the practitioner manipulating the balloon delivery device 608 from the insertion site for the second branch 206) consistent with the expansion/deployment of the second branching portion 112 of the expandable branching stent prosthesis 102 by the balloon 610 of the balloon delivery device 608.

Once the delivery of the balloon delivery device 608 into the second branching portion 112 of the expandable branching stent prosthesis 102 through the sheath 606 of the dilation device 602 is accomplished, the sheath 606 may be retracted out from the branching implant site 202/out of the body altogether.

As illustrated in FIG. 6E, once the balloon delivery device 608 is positioned, the balloon delivery device 608 may be operated to inflate the balloon 610 (e.g., using a control of the balloon delivery device 608). The inflation of the balloon 610 expands the second branching portion 112 of the expandable branching stent prosthesis 102, such that second branching portion 112 deploys against the walls of the second branch 206 of the branching implant site 202. The balloon 610 may then be deflated, and the balloon delivery device 608 removed away from the branching implant site 202/the body altogether.

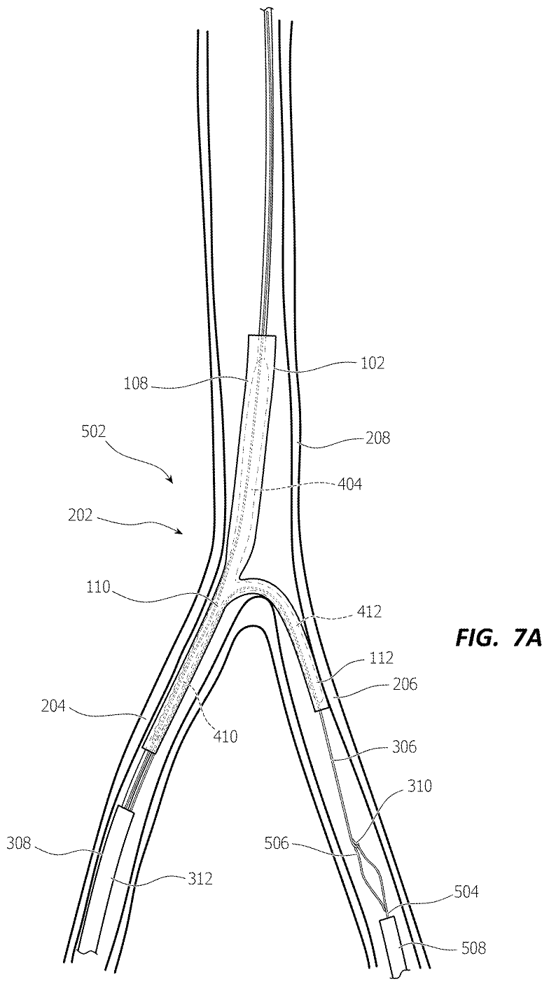

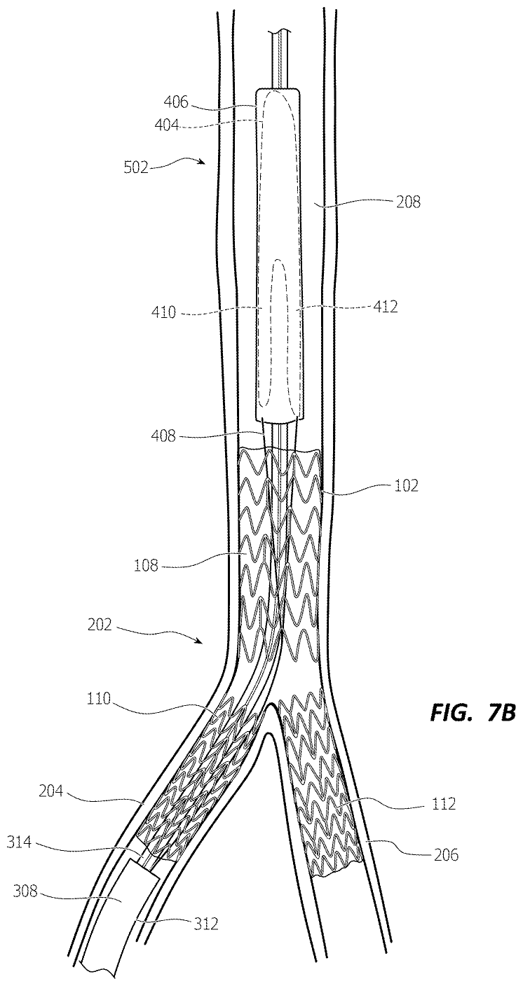

FIG. 7A and FIG. 7B illustrate further subsequent deployment stages for a second deployment system 502, according to embodiments herein. In an alternative to the use of the deployment system 302 of FIG. 3 (e.g., as attendant to embodiments for FIG. 6A through FIG. 6E), it may be instead that the deployment system 402 of FIG. 4 is instead used.

As may be seen with reference to FIG. 4, the balloon 404 of the deployment system 402 is forked such that the (single) inflation of this balloon acts to expand/deploy each of the trunk portion 108, the first branching portion 110, and the second branching portion 112 of the expandable branching stent prosthesis 102 (with the inflation of the second balloon leg 412 of the balloon 404 acting to expand the second branching portion 112 of the expandable branching stent prosthesis 102). Accordingly, various aspects of the serialized deployment stages described in relation to FIG. 6A through FIG. 6E may not be used in such cases.

FIG. 7A illustrates an example positioning of the deployment system 502 once seating of the expandable branching stent prosthesis 102 has occurred. As may be seen, in the present embodiment, the floss wire 306 is not necessarily pulled back along the second branch 206 in order to bring the distal end of the floss wire 306 through the second branch 206 and out through the insertion site/incision for access to the second branch 206, as was described in relation to the embodiments of FIG. 6A through FIG. 6E. Because the inflation of the balloon 404 can deploy the second branching portion 112 of the expandable branching stent prosthesis 102 in addition to the trunk portion 108 and the first branching portion 110 of the expandable branching stent prosthesis 102, it may be ultimately unnecessary to send a follow-up balloon up the second branch 206 of the branching implant site 202. Accordingly, there may be no issue with leaving the floss wire 306 and the snare wire 504 in/around the indicated position within the branching implant site 202 in this case. (Note that in alternative embodiments, if follow-up therapies after the deployment of the expandable branching stent prosthesis 102 would be enabled via the pulling through of the floss wire 306, this may be done in any case, analogous to description in relation to FIG. 6A through FIG. 6E.)

Once the catch feature 310 and the snare feature 506 are snared together, one or more of the floss wire 306 and the and the snare wire 504 may be adjusted, pulled, and/or manipulated, etc. (e.g., by the practitioner using ends of the floss wire 306 and/or the snare wire 504 at the respective insertion site for each) to seat the first branching portion 110 of the expandable branching stent prosthesis 102 along the first branch 204 of the branching implant site 202 and the second branching portion 112 of the expandable branching stent prosthesis 102 along the second branch 206 of the branching implant site 202, in the manner illustrated in FIG. 7A.

With the deployment system 502 in the illustrated position, it may be that the expandable branching stent prosthesis 102 can be usefully deployed within the branching implant site 202. Accordingly, the balloon 404 may be inflated (e.g., the practitioner may manipulate the delivery catheter system 308 for the balloon 404 to cause the inflation of the balloon 404). The inflation of this balloon 404 expands the trunk portion 108, the first branching portion 110, and the second branching portion 112 of the expandable branching stent prosthesis 102, such that they deploy against the walls of the trunk 208, the first branch 204, and the second branch 206 of the branching implant site 202. The balloon 404 may then be deflated.

Note that after the use and subsequent deflation of the balloon 404, removal of the catheter lumen 314 (to which the balloon 404 is attached) back through the branching implant site 202 down the first branch 204 (and, more generally, back out of the body through the insertion site) may become problematic if the second balloon leg 412 of the balloon 404 (which may not be attached along its length to the catheter lumen 314) is allowed to remain free, as the second balloon leg 412 may snag.

FIG. 7B illustrates a socking stage for a deployment system 502 using a forked balloon 404, according to embodiments herein. In order to prevent the balloon 404 from catching when the catheter lumen 314 is being retracted, as illustrated in FIG. 7B, prior to the retraction of the catheter lumen 314 and/or the delivery catheter system 308 down the first branch 204, the catheter lumen 314 and/or the delivery catheter system 308 may be advanced up the trunk 208 while the sock wire(s) 408 is/are held stationary. This causes the sock 406 to unroll (in the downward direction) and be pulled over the balloon 404 while the catheter lumen 314 travels up the trunk 208. Once the sock 406 covers the balloon 404, the catheter lumen 314 and/or the delivery catheter system 308 may be retracted from the branching implant site 202 and/or out of the body back through the insertion site without concern that the second balloon leg 412 of the balloon 404 will snag.

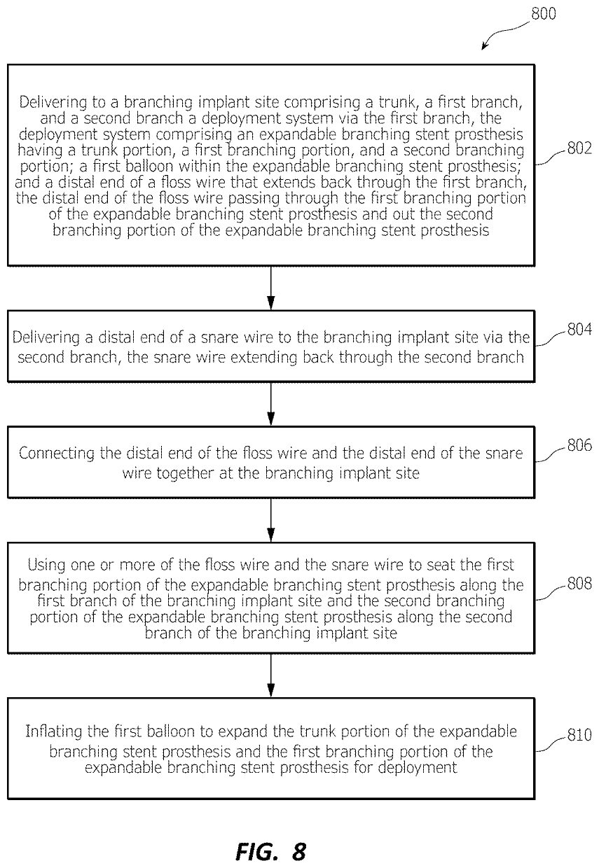

FIG. 8 illustrates a method 800, according to embodiments disclosed herein. The method 800 includes delivering 802 to a branching implant site comprising a trunk, a first branch, and a second branch a deployment system via the first branch, the deployment system comprising an expandable branching stent prosthesis having a trunk portion, a first branching portion, and a second branching portion; a first balloon within the expandable branching stent prosthesis; and a distal end of a floss wire that extends back through the first branch, the distal end of the floss wire passing through the first branching portion of the expandable branching stent prosthesis and out the second branching portion of the expandable branching stent prosthesis.

The method 800 further includes delivering 804 a distal end of a snare wire to the branching implant site via the second branch, the snare wire extending back through the second branch.

The method 800 further includes connecting 806 the distal end of the floss wire and the distal end of the snare wire together at the branching implant site.

The method 800 further includes using 808 one or more of the floss wire and the snare wire to seat the first branching portion of the expandable branching stent prosthesis along the first branch of the branching implant site and the second branching portion of the expandable branching stent prosthesis along the second branch of the branching implant site.

The method 800 further includes inflating 810 the first balloon to expand the trunk portion of the expandable branching stent prosthesis and the first branching portion of the expandable branching stent prosthesis for deployment.

In some embodiments, the method 800 further includes pulling the snare wire to bring the distal end of the floss wire through the second branch and out of an access incision of the second branch; delivering, along the floss wire, through the first branching portion of the expandable branching stent prosthesis, into the second branching portion of the expandable branching stent prosthesis, a dilation device that dilates the second branching portion of the expandable branching stent prosthesis; delivering, along the floss wire, into the second branching portion of the expandable branching stent prosthesis, a second balloon; and inflating the second balloon to expand the second branching portion of the expandable branching stent prosthesis for deployment.

In some embodiments of the method 800, the inflating the first balloon further expands the second branching portion of the expandable branching stent prosthesis for deployment. In some such embodiments, the deployment system further comprises a sock configured to be pulled over the balloon, and the method 800 further includes deflating the first balloon and pulling the sock over the first balloon. In some of these cases, the sock comprises PTFE.

In some embodiments of the method 800, the branching implant site is within a vascular system.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

References to approximations are made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially perpendicular” is recited with respect to a feature, it is understood that in further embodiments, the feature can have a precisely perpendicular configuration.

Similarly, in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A method comprising:
delivering to a branching implant site comprising a trunk, a first branch, and a second branch a deployment system via the first branch, the deployment system comprising:
an expandable branching stent prosthesis having a trunk portion, a first branching portion, and a second branching portion,
a first balloon within the expandable branching stent prosthesis, and
a distal end of a floss wire that extends back through the first branch, the distal end of the floss wire passing through the first branching portion of the expandable branching stent prosthesis and out the second branching portion of the expandable branching stent prosthesis;
delivering a distal end of a snare wire to the branching implant site via the second branch, the snare wire extending back through the second branch;
connecting the distal end of the floss wire and the distal end of the snare wire together at the branching implant site;
using one or more of the floss wire and the snare wire to seat the first branching portion of the expandable branching stent prosthesis along the first branch of the branching implant site and the second branching portion of the expandable branching stent prosthesis along the second branch of the branching implant site; and
inflating the first balloon to expand the trunk portion of the expandable branching stent prosthesis and the first branching portion of the expandable branching stent prosthesis for deployment.

2. The method of claim 1, further comprising:
pulling the snare wire to bring the distal end of the floss wire through the second branch and out of an access incision of the second branch;
delivering, along the floss wire, through the first branching portion of the expandable branching stent prosthesis, into the second branching portion of the expandable branching stent prosthesis, a dilation device that dilates the second branching portion of the expandable branching stent prosthesis;
delivering, along the floss wire, into the second branching portion of the expandable branching stent prosthesis, a second balloon; and
inflating the second balloon to expand the second branching portion of the expandable branching stent prosthesis for deployment.

3. The method of claim 1, wherein the inflating the first balloon further expands the second branching portion of the expandable branching stent prosthesis for deployment.

4. The method of claim 3, wherein the deployment system further comprises a sock configured to be pulled over the balloon, and further comprising:
deflating the first balloon; and
pulling the sock over the first balloon.

5. The method of claim 4, wherein the sock comprises polytetrafluoroethylene (PTFE).

6. The method of claim 1, wherein the branching implant site is within a vascular system.

7. A deployment system for deployment by a deployment device to a branching implant site having a trunk, a first branch, and a second branch, comprising:
an expandable branching stent prosthesis having a trunk portion, a first branching portion, and a second branching portion;
a balloon within the expandable branching stent prosthesis; and
a distal end of a floss wire, the distal end of the floss wire passing through the first branching portion of the expandable branching stent prosthesis and out the second branching portion of the expandable branching stent prosthesis.

8. The deployment system of claim 7, wherein the distal end of the floss wire terminates in a catch.

9. The deployment system of claim 7, wherein the balloon is configured to inflate to deploy the trunk portion of the expandable branching stent prosthesis and the first branching portion of the expandable branching stent prosthesis.

10. The deployment system of claim 9, wherein the balloon is further configured to inflate to deploy the second branching portion of the expandable branching stent prosthesis.

11. The deployment system of claim 10, further comprising a sock configured to be pulled over the balloon.

12. The deployment system of claim 11, wherein the sock comprises polytetrafluoroethylene (PTFE).

13. The deployment system of claim 7, wherein the branching implant site is within a vascular system.

14. A system for use with a branching implant site having a trunk, a first branch, and a second branch, the system comprising:
an expandable branching stent prosthesis having a trunk portion, a first branching portion, and a second branching portion;
a balloon within the expandable branching stent prosthesis;
a distal end of a floss wire that passes through the first branching portion of the expandable branching stent prosthesis and out the second branching portion of the expandable branching stent prosthesis; and
a catheter lumen configured to support the distal end of the floss wire, the expandable branching stent prosthesis, and the balloon during a delivery of the distal end of the floss wire, the expandable branching stent prosthesis, and the balloon to the branching implant site.

15. The system of claim 14, further comprising a catheter sleeve configured to surround the distal end of the floss wire, the expandable branching stent prosthesis, the balloon, and the catheter lumen during the delivery.

16. The system of claim 14, wherein the distal end of the floss wire terminates in a catch.

17. The system of claim 14, wherein the balloon is configured to inflate to deploy the trunk portion of the expandable branching stent prosthesis and the first branching portion of the expandable branching stent prosthesis.

18. The system of claim 17, wherein the balloon is further configured to inflate to deploy the second branching portion of the expandable branching stent prosthesis.

19. The system of claim 18, further comprising a sock configured to be pulled over the balloon.

20. The system of claim 19, further comprising a catheter sleeve configured to surround the distal end of the floss wire, the expandable branching stent prosthesis, the balloon, the catheter lumen, and the sock during the delivery.
